(19) United States
(12) Patent Application Publication
(10) Pub. No.: US 2026/0283647 A1
(43) Pub. Date: Sep. 24, 2026

(54) DEVICES, SYSTEMS AND METHODS FOR TREATING THE SKIN

(71) Applicant: HydraFacial LLC, Long Beach, CA (US)

(72) Inventor: Haroon Lais, Chino Hills, CA (US)

(21) Appl. No.: 19/474,157

(22) PCT Filed: Apr. 11, 2024
(86) PCT No.: PCT/US2024/024139

§ 371 Date: Oct. 9, 2025

Related U.S. Application Data
(60) Provisional application No. 63/458,927, filed on Apr. 12, 2023.

(51) Int. Cl. A61B 17/54; A61B 34/00

(52) U.S. Cl. CPC A61B 17/545; A61B 34/25; A61B 2217/005

(57) ABSTRACT

According to some embodiments, a skin treatment system comprises at least two stations, each of the at least two stations being configured to receive a fluid container, wherein the at least two stations are included in a manifold assembly, a single fully-enclosed collection header, and at least one fluid connector configured to place treatment material included in the fluid containers secured to the stations in fluid communication with the collection header, wherein a downstream end of the collection header is configured to be in fluid communication with a fluid conduit that places the collection header in fluid communication with a handpiece.

### CROSS-REFERENCE TO RELATED APPLICATIONS

This application claims the priority benefit of U.S. Provisional Patent Application No. 63/458927, filed Apr. 12, 2023, the contents of which are incorporated by reference herein in their entirety.

### FIELD

This application relates generally to skin treatment, and more specifically, to apparatuses, systems and methods for treating a person's skin.

### DESCRIPTION OF THE RELATED ART

Abrasion of the outer layer or epidermis of the skin is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

### SUMMARY

According to some embodiments, a skin treatment system comprises at least two stations, each of the at least two stations being configured to receive a fluid container, wherein the at least two stations are included in a manifold assembly or other fluid distribution system, a single fully-enclosed collection header, and at least one fluid connector configured to place treatment material included in the fluid containers secured to the stations in fluid communication with the collection header, wherein a downstream end of the collection header is configured to be in fluid communication with a fluid conduit that places the collection header in fluid communication with a handpiece.

According to some embodiments, a skin treatment system comprises at least two stations, each of the at least two stations being configured to receive a fluid container, wherein the at least two stations are included in a manifold assembly or other fluid distribution system, a single fully-enclosed collection header, and at least one fluid connector configured to place treatment material included in the fluid containers secured to the stations in fluid communication with the collection header, wherein a downstream end of the collection header is configured to be in fluid communication with a fluid conduit that places the collection header in fluid communication with a handpiece.

According to some embodiments, the collection header is machined from a larger member comprising at least one metal and/or alloy (e.g., aluminum, stainless steel, etc.).

According to some embodiments, the collection header is configured to be placed in fluid communication with a purge system. In some embodiments, an upstream end of the collection header is configured to be placed in fluid communication with a purge system.

According to some embodiments, the purge system is configured to permit the collection header to be selectively purged or substantially purged of any treatment material. In some embodiments, the purge system comprises a valve or other flow control device that is configured to place the collection header in fluid communication with an ambient space.

According to some embodiments, the purge system comprises a pump, compressor or other device configured to create a positive pressure relative to an ambient pressure within the collection header. In other embodiments, the purge system does not comprise a pump, compressor or other device configured to create a positive pressure relative to an ambient pressure within the collection header.

According to some embodiments, the system further comprises a vacuum source, wherein the vacuum source is configured to create a vacuum or negative pressure relative to an ambient pressure within the collection header.

According to some embodiments, the manifold assembly is included in a tower or console of the system. In some embodiments, the system further comprises a user interface (e.g., a touchscreen device or component).

According to some embodiments, the system further comprises a handpiece configured to be placed in fluid communication with the collection header and other portions of the manifold assembly. In some embodiments, the manifold assembly comprises at least three stations (e.g., 3, 4, more than 5, etc.).

According to some embodiments, the collection header comprises a circular or oval cross-sectional shape.

According to some embodiments, the collection header comprises a single conduit or passage. In some embodiments, an interior of the collection header comprises at least one layer, coating, lining or member.

According to some embodiments, the at least one layer, coating, lining or member reduces friction of fluid flow through the collection header, offers protection to the collection header and/or provides one or more benefits or advantages.

According to some embodiments, the at least one layer, coating, lining or member is hydrophobic (e.g., comprises a hydrophobic coating or layer). In some embodiments, the at least one layer, coating, lining or member comprises at least one of the following: polytetrafluoroethylene (PTFE), perfluoroalkoxy alkanes (PFA), fluorinated ethylene propylene (FEP), another fluoropolymers, acetal, another synthetic fluoropolymer of tetrafluoroethylene and/or any polymer or material.

According to some embodiments, the at least one layer, coating, lining or member comprises an insert that is removably or fixedly attached to an interior of the collection header. In some embodiments, the at least one layer, coating, lining or member is deposited or otherwise provided along an interior of the collection header at the time of manufacture of the system.

According to some embodiments, a skin treatment system comprises at least two stations, each of the at least two stations being configured to receive a fluid container, wherein the at least two stations are included in a manifold assembly or other fluid distribution system, a collection header, wherein treatment material included in the fluid containers secured to the stations is in fluid communication with the collection header, and wherein a downstream end of the collection header is configured to be in fluid communication with a fluid conduit that places the collection header in fluid communication with a handpiece. The skin treatment system further comprises a purge system configured to permit the collection header to be selectively purged or substantially purged of any treatment material.

According to some embodiments, an upstream end of the collection header is configured to be placed in fluid communication with a purge system.

According to some embodiments, the purge system comprises a valve or other flow control device that is configured to place the collection header in fluid communication with an ambient space.

According to some embodiments, the purge system comprises a pump, compressor or other device configured to create a positive pressure relative to an ambient pressure within the collection header. In some embodiments, the purge system does not comprise a pump, compressor or other device configured to create a positive pressure relative to an ambient pressure within the collection header.

According to some embodiments, the system further comprises a vacuum source, wherein the vacuum source is configured to create a vacuum or negative pressure relative to an ambient pressure within the collection header.

According to some embodiments, the manifold assembly is included in a tower or console of the system. In some embodiments, the system further comprises a user interface (e.g., a touchscreen device or component).

According to some embodiments, the system further comprises a handpiece configured to be placed in fluid communication with the collection header and other portions of the manifold assembly.

According to some embodiments, the collection header comprises a single fully-enclosed collection header.

According to some embodiments, the collection header is machined from a larger member comprising at least one metal and/or alloy (e.g., aluminum, stainless steel, etc.).

According to some embodiments, the collection header comprises a single conduit or passage. In some embodiments, an interior of the collection header comprises at least one layer, coating, lining or member.

According to some embodiments, the at least one layer, coating, lining or member reduces friction of fluid flow through the collection header, offers protection to the collection header and/or provides one or more benefits or advantages.

According to some embodiments, the at least one layer, coating, lining or member is hydrophobic (e.g., comprises a hydrophobic coating or layer). In some embodiments, the at least one layer, coating, lining or member comprises at least one of the following: polytetrafluoroethylene (PTFE), perfluoroalkoxy alkanes (PFA), fluorinated ethylene propylene (FEP), another fluoropolymers, acetal, another synthetic fluoropolymer of tetrafluoroethylene and/or any polymer or material.

According to some embodiments, the at least one layer, coating, lining or member comprises an insert that is removably or fixedly attached to an interior of the collection header. In some embodiments, the at least one layer, coating, lining or member is deposited or otherwise provided along an interior of the collection header at the time of manufacture of the system.

### BRIEF DESCRIPTION OF THE DRAWINGS

These and other features, aspects and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 schematically illustrates a flow diagram for a skin treatment system having a manifold assembly according to one embodiment;

FIG. 2 illustrates a perspective view of a console or tower of a skin treatment system according to one embodiment;

FIG. 3A schematically illustrates a flow diagram for a skin treatment system comprising a manifold assembly according to one embodiment;

FIG. 3B schematically illustrates a flow diagram for a skin treatment system comprising a manifold assembly according to another embodiment;

FIG. 4 schematically illustrates a side cross-sectional view of distal end of a handpiece assembly and a tip configured to secure thereto according to one embodiment;

FIGS. 5A and 5B schematically illustrate side cross-sectional views of distal end of a handpiece assembly and a sealing cap configured to secure thereto according to one embodiment;

FIG. 5C illustrates a side view of handpiece with a sealing cap secured to its distal end according to one embodiment;

FIG. 6 illustrates a perspective view of a manifold assembly having a purge system according to one embodiment;

FIG. 7 illustrates a top view of a collection zone or portion of a manifold assembly according to one embodiment;

FIG. 8A illustrates a cross-sectional view of the collection zone or portion of FIG. 7 according to one embodiment;

FIG. 8B illustrates a cross-sectional view of the collection zone or portion of FIG. 7 according to another embodiment; and

FIGS. 9A to 9C schematically illustrate various embodiments of fluid flow schemes using a manifold assembly having a purge system.

### DETAILED DESCRIPTION

Although the various embodiments of a handpiece assembly have specific relevance to a skin treatment system, the features, advantages and other characteristics disclosed herein may have direct or indirect applicability in other applications, such as, for example, medical devices, mechanical devices and/or the like.

Several embodiments of the inventions disclosed herein are particularly advantageous because they include one, several or all of the following benefits: provide for enhanced delivery of treatment fluids to the skin of a subject; provide for delivery of fluids to the skin of a subject while reducing the likelihood of contamination and/or reducing the amount of treatment fluid that is wasted or otherwise not delivered to the skin surface being treated; protect one or more components of the manifold assembly; prevent or reduce the likelihood of leaks in a manifold assembly.

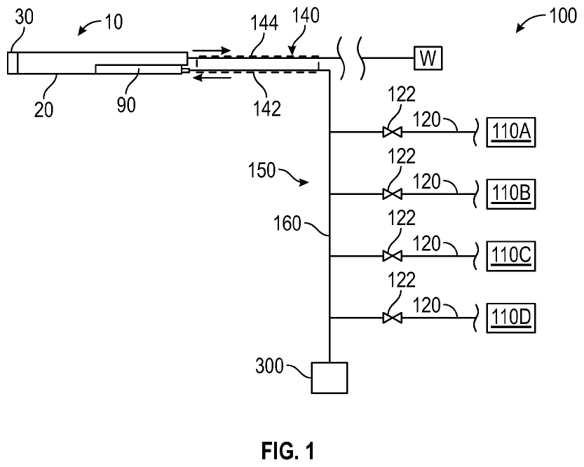

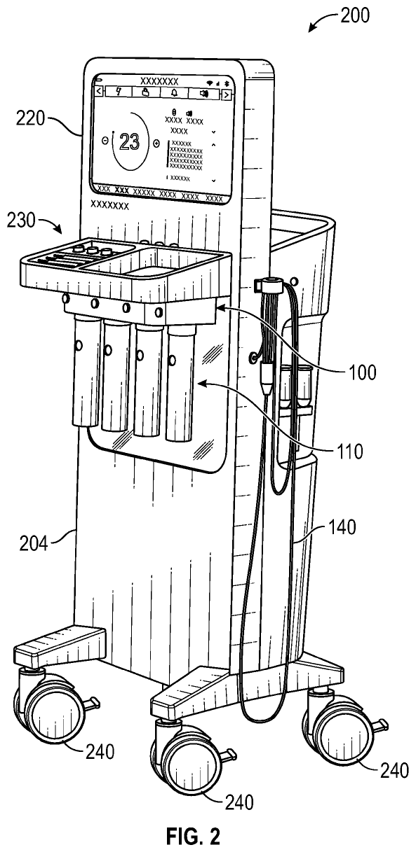

According to certain embodiments, as schematically illustrated in FIG. 1, a skin treatment system can include a manifold assembly or other fluid distribution member, system or sub-system 100 that facilitates the transfer of fluids and/or other treatment materials from one or more bottles or other containers 110A, 110B, 110C, 110D secured to a handpiece 10 (or other device configured to contact skin and/or provide treatment to skin). As illustrated in FIG. 2, in some embodiments, the manifold assembly 100 can be incorporated, at least partially in (e.g., partially, completely, etc.) a tower or console system or device 200.

In some arrangements, the bottles, canisters or other containers 110 that are designed and otherwise adapted to be secured to the tower or console 200 can be replaceable, interchangeable and/or otherwise removable (e.g., for replacement, emptying, autoclaving or other types of cleaning, etc.).

With continued reference to the embodiment of FIG. 2, the tower or console 200 can include various components and features. For example, in some configurations, the tower 200 comprises a manifold assembly 100 for receiving one or more (e.g., 1, 2, 3, 4, more than 4, etc.) bottles or other containers 110 of treatment fluids to be used in a skin treatment procedure. The tower or console 200 can further include one or more waste canisters or other containers that are configured to receive spent fluids, exfoliated or otherwise removed skin tissue and/or other waste products resulting from a skin treatment procedure, a vacuum or suction pump or other vacuum or suction source, a user interface or user input 220 (e.g., a touchscreen, keyboard, other input device, monitor or other output device or component, etc.), an outer housing or other exterior structure 204, tubing, one or more trays or other storage components 230, casters or other wheels 240, interior components (e.g., processor, memory, power source, sensors, tubing or other fluid conduits, channels and/or passages, valves, other flow control devices and/or other hydraulic components, electrical wiring and other electrical components, etc.) and/or the like.

In some embodiments, the console includes a gesture control system. A gesture control system can be configured to permit a user to modify or otherwise control or regulate at least one aspect of the operation of the system. For example, a user can modify (e.g., increase or decrease) a vacuum or suction pressure, modify (e.g., increase or decrease) a rate of flow or delivery of treatment material to the distal end of the handpiece (e.g., by controlling vacuum force, by regulating one or more valves or other flow control device, etc.) and/or modify any other aspect of the system. According to some embodiments, the system is configured to receive at least one command from a user using a gesture control system. In some embodiments, the gesture control system comprises at least two gesture sensors. In one embodiment, the gesture control system comprises at least four gesture sensors. Additional details regarding gesture control concepts that can be incorporated into any of the embodiments disclosed herein or equivalents thereof are included in PCT Publication WO 2023/064718, filed Oct. 7, 2022 as PCT Appl. PCT/US 2022/077811 and published on Apr. 20, 2023. The entire contents of WO 2023/064718 are incorporated by reference herein and made a part of the present application.

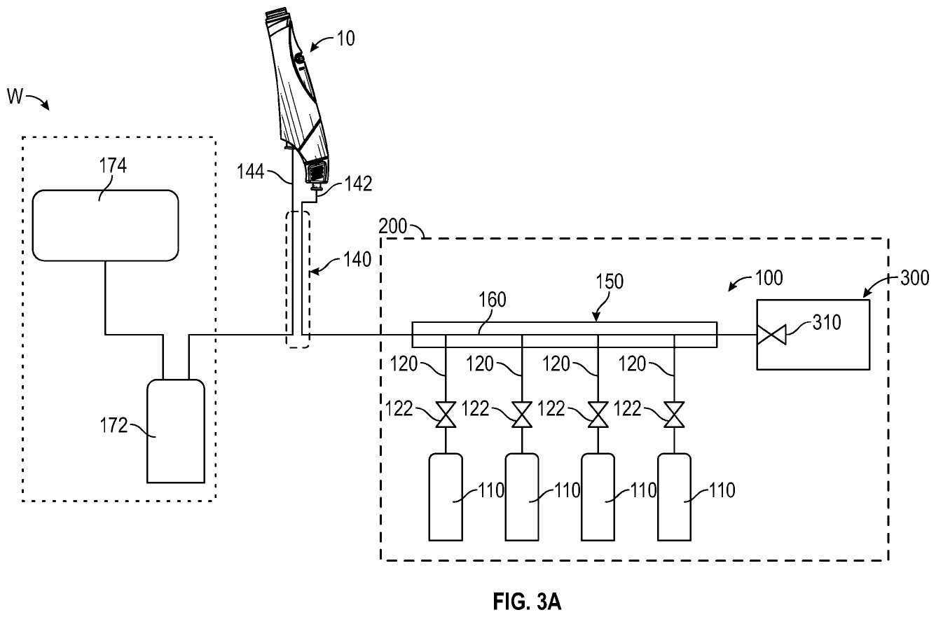

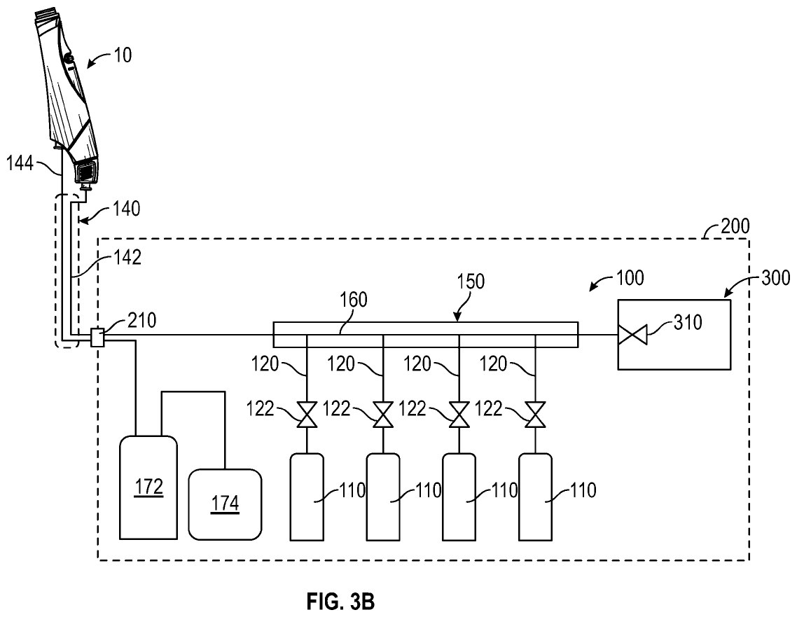

As illustrated schematically in FIG. 1, one or more valves or other flow control devices or components 122 can be positioned between each fluid source (e.g., a bottle) 110A, 110B, 110C, 110D and a main delivery conduit or other fluid line 142 that places the manifold assembly 100 in fluid communication with a handpiece 10. In some arrangements, as schematically illustration in FIGS. 1, 3A and 3B, the treatment fluid/material delivery conduit or line 142 can be included as part of and/or within a multi-lumen conduit 140 that fluidly connects the handpiece 10 with the manifold assembly 100. For example, such a multi-lumen conduit 140 can include one or more lumens or passages (e.g., treatment fluid/material delivery passages) 142 that are configured to transfer treatment fluids and/or other treatment materials from the manifold assembly 100 to the handpiece 10 and one or more fluidly distinct lumens or passages (e.g., waste or suction passages) 144 that are configured to transfer waste (e.g., spent fluids, exfoliated skin, sebum, blackheads, etc.) from or near the tip 30 to a waste container. In other embodiments, however, the treatment fluid delivery passages 142 are included in a separate conduit or device than the waste or suction passages 144, as desired or required.

In FIG. 1, the manifold can include valves or other flow control devices 122 that are configured to regulate the flow of treatment fluids and/or other materials from one or more of the fluid sources (e.g., bottles, other containers, etc.) 110A, 110B, 110C, 110D to the main delivery conduit 142, and thus, to the handpiece 10. In some arrangements, such valves 122 comprise 2-way valves. However, any other type of valve or other fluid control device can be used.

As depicted schematically in FIG. 1, the handpiece 10 can include or can be configured to receive a cartridge 90. The cartridge 90 can include a port or other connector that can be placed in fluid communication with the main delivery conduit 142. Such a cartridge 90 can be fixedly or removably secured to and/or incorporated into (e.g., directly or indirectly) the handpiece 10. For example, in some embodiments, the cartridge 90 includes a “dummy” cartridge that is removably secured within a recess, slot or other opening of the handpiece 10 (e.g., a recess that extends to an exterior of the housing 20 of the handpiece).

With continued reference to FIG. 1, the handpiece 10 can be placed in fluid communication with a waste system W. The waste system W can include, without limitation, one or more of the following devices, components and/or features: a suction source (e.g., vacuum pump), a waste canister or other waste container, one or more fluid conduits, valves and/or other hydraulic components or features and/or the like. Regardless of its exact configuration, in some embodiments, the waste system W includes or is configured to be placed in fluid communication with one or more waste conduits or lines 144. Such a waste conduit or line 144 can be configured to secure to a waste coupling or port of the handpiece assembly 10. In some arrangements, the waste coupling or port is located at or near a proximal end of the handpiece 10. However, in alternative embodiments, the waste coupling or port is located along a side or other exterior surface of the housing 20 of the handpiece 10, as desired or required.

In the arrangements disclosed herein, the manifold assembly 100 comprises three or four fluid branches. However, a system can comprise any number of fluid branches (e.g., 1, 2, 3, 4, 5, 6, 7, 8, more than 8, etc.), as desired or required by a particular application or use. Further, one or more of the bottles, other containers or other fluid sources 110A, 110B, 110C, 110D that are positioned within (e.g., secured to) the manifold assembly 100 can include a cleaning and/or rinsing solution (e.g., for the periodic cleaning and/or flushing of the manifold assembly and/or other components with which a treatment fluid or other material may come in contact).

According to certain embodiments, one or more of the treatment fluid delivery conduits 142 are configured to receive and transfer (e.g., toward the handpiece) a serum, other treatment fluid, material and/or the like. Alternatively or in addition, one or more of the fluid delivery conduits 142 can be configured to receive and transfer (e.g., toward the handpiece) water (e.g., distilled, tap water, filtered, etc.), saline, other dilutants or dissolvents, other fluids and/or the like to the handpiece 10. Such fluids can be adapted to, at least partially, contact, dissolve, dilute, liquefy, soften and/or otherwise mix with one or more solids, gels and/or other materials positioned within or on various surfaces or portions of the handpiece 10 (e.g., tip 30 of the handpiece or secured to a distal end of the handpiece) and/or other treatment fluids and/or materials present or delivered (e.g., via the manifold assembly) to or near the tip-skin surface interface. In some arrangements, such fluid schemes can provide a convenient way of delivering one or more materials at the skin-tip interface and/or any other location where such materials are desired or required.

With continued reference to the schematic of FIG. 1, the manifold assembly 100 can include individual conduits, passages or other lines 120 that place the contents of each bottle, other container or other fluid source 110A, 110B, 110C, 110D secured to the assembly 100 in fluid communication with a collection portion or zone 150 of the assembly 100. In some arrangements, the collection portion or zone 150 comprises a collection passage or header 160 that is fluid communication with all individual conduits or passages 120 of the assembly. Such an embodiment can include a parallel fluid configuration. In other embodiments, the collection portion or zone 150 can include two or more collection passages or headers 160, as desired or required. As shown schematically in FIGS. 1, 3A, 3B and 6 and discussed herein, one or more (e.g., some or all of) the individual conduits or passages 120 can include at least one valve or other flow control device 122. The valves or other flow control devices 122 can be adapted to regulate the flow of fluids (e.g., treatment materials, water, rinsing solution, other cleaning fluids, etc.) and/or other materials through the corresponding individual conduits or passages 120 during use of the skin treatment system. In some embodiments, the valves 122 are configured to be manually and/or automatically controlled. In some configurations, such control occurs via remote control (e.g., using solenoids and/or other devices, components or features). However, localized control of the valves or other flow control devices 122 can also be used, either in addition to or in lieu of remote control.

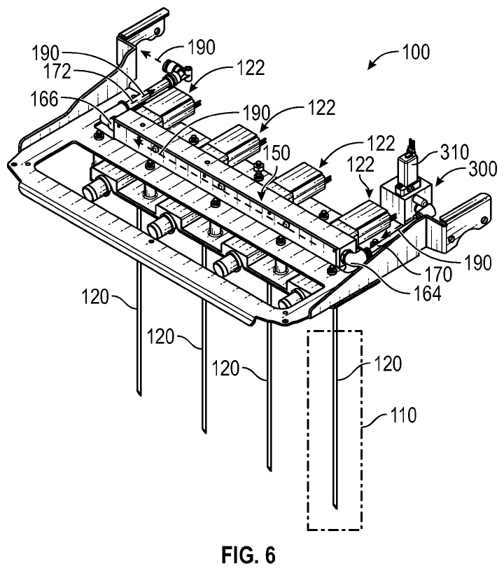

As illustrated in FIG. 1, fluids and/or other materials being transferred through each individual conduit or passage 120 can be delivered to a common or collection conduit, channel, header or passage 160 along the collection portion or zone 150. Such individual conduits or passages 120 can comprise tubes or similar components or features that are configured to extend into the interior of bottles or other containers 110A, 110B, 110C, 110D secured to the manifold assembly 100. See, for example, the embodiment of a manifold assembly 100 depicted in FIG. 6 and discussed further below. Such individual conduits or passages 120 can be configured to extend to or near the bottom of the interior of the bottles or other containers 110A, 110B, 110C, 110D. In some embodiments, the valves 122 can be positioned at any location between the fluid source (e.g., bottle or container) 110A, 110B, 110C, 110D and the collection conduit, channel or passage 160. Such valves can include any type of valve and can be configured to be operated manually or automatically, as desired or required. In some embodiments, the valves can include a proportional valve (e.g., electronically triggered/actuated via a solenoid or similar device, component or feature).

As discussed herein, components of skin treatment system can be included within and/or on a console or tower 200 (see, e.g., FIG. 2). The system can be configured to include and/or to be used with one or more handpieces (e.g., handpieces, other handheld devices, etc.) 10, as desired or required. The system can include a waste system W which comprises a suction or vacuum source (e.g., vacuum pump). In some embodiments, the suction or vacuum source and/or other portions of the waste system W are included with the system; however, in other arrangements, the system is configured to selectively couple to a suction or vacuum source (e.g., a suction or vacuum source that is separate of the system) and/or other portions of the waste system W.

According to some embodiments, once activated, the vacuum source of the waste system W can generate suction (e.g., negative pressure relative to atmospheric or ambient pressure) along the distal end (e.g., the tip) 30 of a handpiece 10. In some arrangements, such a suction or vacuum force generated along the tip or distal end 30 of a handpiece helps draw or “pull” one or more treatment fluids to the tip or distal end (e.g., via a manifold assembly 100, a fluid conduit 142, etc.). In some embodiments, the tip or distal end 30 of the handpiece 10 includes a peripheral lip or member that is configured to contact skin tissue. Such a peripheral lip or member can extend continuously around the distal end of the tip. In other arrangements, however, the peripheral lip or member extends only partially around the distal end of the tip.

During use, when the aesthetician or other user places the tip or distal end of the handpiece along a subject's skin surface to be treated, the lip or other peripheral member can form at least a partial seal with the targeted skin surface and help generate a vacuum or suction force within an interior area circumscribed by the lip or member. In turn, according to some arrangements, the vacuum or suction force along such an interior area along the tip or distal end 30 can help draw fluids and/or other treatment materials from one or more fluid sources 110A, 110B, 110C, 110D (e.g., bottles or other containers secured to the manifold) to the tip or distal end (e.g., using the suction or vacuum alone without the need for positive pressure exerted on the fluids or other treatment materials being transferred to the tip or distal end).

According to certain embodiments, as schematically illustrated in FIG. 1, one or more bottles or other containers 110A, 110B, 110C, 110D can be placed in fluid communication with a manifold assembly 100 or other multi-branch assembly or system. As discussed above, in some arrangements, as depicted schematically in FIG. 3A, one or more valves or other flow control devices or components 122 can be positioned between each fluid source (e.g., a bottle) and a main fluid delivery conduit or other fluid line 142 that places the manifold assembly 100 in fluid communication with a handpiece 10. The valves or other flow control devices 122, which can include 2-way valves, can be configured to regulate the flow of treatment materials from one or more of the bottles or other containers 110A, 110B, 110C, 110D to the main delivery conduit 142, and thus, to the handpiece 10.

For any of the embodiments disclosed herein, a handpiece 10 the fluid delivery conduit(s) or other fluid line(s) 142 (e.g., which selectively deliver(s) fluids and/or other materials from one or more fluid sources 110 secured to the manifold assembly 100) and the waste conduit(s) or other waste line(s) 144 (e.g., which transfer(s) spent fluids, exfoliated skin, oils, sebum, blackheads and other materials removed from the skin surface being treated and the like to a waste container with the assistance of a vacuum or suction source of the waste system W) can be integrated into a single conduit 140. Such combined tubing or other conduit 140 can include a multi-lumen tubing or other conduit. In other embodiments, such fluid and waste lines 142, 144 can be separate conduits that are secured or connected (at least partially, e.g., continuously, intermittently, etc.) for a particular length. In yet other arrangements, the fluid delivery and waste conduits or lines 142, 144 are separate from each other and are not coupled or otherwise connected, as desired or required.

As depicted schematically in FIG. 1 and discussed in detail herein, the manifold assembly 100 can include a purge assembly, device, element, system and/or component 300. Such a purge assembly or system 300 can be configured to advantageously transfer fluids and/or other treatment materials remaining within the manifold (e.g., the collection conduit, passage or header 160) and/or other components in fluid communication with the manifold 100 to the fluid delivery conduit 142 and the handpiece 10.

The use of a purge assembly or system 300 can provide one or more benefits to the manifold assembly, and thus to the overall skin treatment system. For instance, the mixing or other cross-contamination of different treatment fluids and/or other fluids contained within the various bottles or other containers 110 secured to the manifold assembly 110A, 110B, 110C, 110D is reduced or eliminated. Further, the volume or amount of treatment fluids, other fluids (e.g., cleaning solutions) and/or other materials transferred from the bottles or other containers 110A, 110B, 110C, 110D that is wasted (e.g., not delivered to the subject being treated, to the handpiece, etc.) is advantageously reduced or eliminated. This can lead to significant cost savings to the user over time especially if a treatment fluid or materials container in a bottles or other container 110A, 110B, 110C, 110D secured to the manifold assembly 100 is relatively expensive and/or difficult to obtain.

As discussed in greater detail below, the purge assembly or system 300 can be positioned along a proximal or upstream end of the manifold assembly 100. The purge assembly 300 can be integrated into the manifold assembly 100 (e.g., manufactured with the manifold assembly 100). Alternatively, the purge assembly or system 300 is separate from the manifold assembly and/or can be detached or decoupled from the manifold assembly, as desired or required. Accordingly, in some embodiments, a purge assembly or system 300 can be provided as a separate apparatus or system that is coupled (e.g., mechanically, fluidly, etc.) to a manifold assembly 100 (e.g., as an add-on, as an after-market product, etc.).

The purge assembly 300 can be configured to purge or clear out fluids and/or other materials from the manifold collection conduit or passage 160 (and/or any other channel, passage or conduit of the manifold system 100) actively or passively. For example, the purge assembly or system 300 can actively purge fluids from the manifold assembly 100 by generating a positive pressure (e.g., pressure greater than ambient pressure, pressure greater than the localized pressure within the manifold assembly, etc.) within the manifold assembly 100 (e.g., the manifold collection conduit or passage 160). Such positive pressure can be created by, at least in part, a compressor, a pump and/or other pneumatic device, component or system.

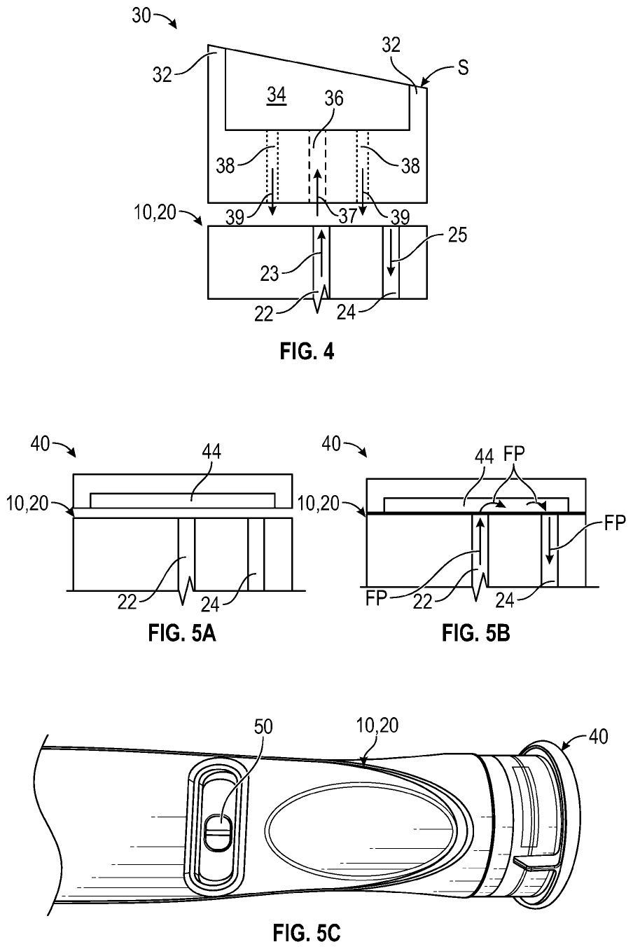

In some embodiments, the purge assembly or system 300 is configured to passively clear one or more conduits, channels or other passages of the manifold assembly 100 (e.g., the manifold collection conduit or passage 160) either in addition to or in lieu of any passive purging arrangements. For example, the purge system 300 can include one or more valves or other flow control devices, components or features 310 that selectively open (e.g., expose) and close the purge assembly 300 to/from the ambient surroundings (e.g., the airspace in the room in which the skin treatment system is situated). In passive configurations of a purge system 300, the driving force necessary to clear or otherwise move fluids and/or other materials positioned within the manifold assembly can be provided by one or more other pressure or vacuum generating devices, components or systems. One such passive embodiment, which is described in greater detail below in connection to FIGS. 4, 5A and 5B, advantageously uses the suction or vacuum created by a suction or vacuum source (e.g., of a waste system W) to generate a suction or vacuum force within the manifold. For example, the suction or vacuum force can be generated along the waste or vacuum conduit 144, the fluid delivery conduit 142, a region at or along the handpiece 10 (e.g., along the distal end of the tip or the distal end of the handpiece) where the waste or vacuum conduit and the fluid delivery conduit are in fluid communication with one another, the collection conduit or header 160 of the manifold assembly 100 and/or any other component or portion of the manifold assembly 100 or the larger treatment system, as desired or required.

Regardless of its exact configuration and other details, the purge system or assembly 300 can advantageously enable treatment fluids and/or other materials being delivered from the manifold assembly 100 to the handpiece (e.g., from one of the bottles or other containers 110A, 110B, 110C, 110D secured to the manifold assembly to the tip 30 of the handpiece 10 via one or more conduits, channels or passages 120, 160, 142) to be completely or substantially completely evacuated or transferred. Accordingly, as noted herein, the use of such a purge system can provide one or more advantages or benefits to the user, the subject being treated and/or the overall skin treatment procedure. Some of these are discussed in the paragraphs that follow.

The purge assembly or system 300 can lower the volume or amount of treatment fluid or other materials that is wasted during use. For instance, one way that fluids and/or other materials can be eliminated without a purge system involves transferring the volume of treatment fluid remaining in the system (e.g., from the bottles or other fluid sources 110A, 110B, 110C, 110D to the handpiece 10) to a waste system W. The wasted volume is what remains in the hydraulic network (e.g., from the bottles or other fluid sources 110A, 110B, 110C, 110D to the handpiece 10) after the desired amount of such fluid has been delivered to the subject's skin. In other words, without a purge system as described herein, the volume of treatment fluid remaining in the hydraulic network of a skin treatment system (e.g., after a desired volume of such a fluid has already been delivered to the skin) needs to be eliminated from the network (e.g., manifold, fluid delivery conduit 142, etc.) but is not actually used in the treatment procedure. As discussed herein, embodiments with a purge assembly or system can permit the desired amount of treatment fluids and/or other materials to be transferred from a skin treatment system's hydraulic network to the skin without the need to waste (e.g., prior to the delivery of a subsequent fluid or material, prior to the termination of a procedure, etc.).

The use of a purge system can also prevent or lower the likelihood of cross-contamination between the various treatment fluids or materials that are positioned within distinct bottles or other containers 110A, 110B, 110C, 110D secured to the manifold assembly 100. Since the purge system 300 advantageously enable for complete or substantially complete transfer of the fluids contained in the system's hydraulic network to the tip of the handpiece, exposure of a previously-delivered treatment fluid or material will not contaminate or contact a subsequently-delivered treatment fluid or material. This can result in a more efficacious and safer procedure for the subject being treated.

FIG. 3A schematically illustrates one embodiment of a skin treatment system that includes a purge assembly, device, system, component or feature 300. As shown, the skin treatment system can comprise a manifold assembly 100. In some embodiments, as previously discussed, such a manifold assembly 100 is positioned entirely or partially within a tower or console 200. In the depicted arrangement, a waste system or portion W of the skin treatment system is separate from the tower or console 200. The waste system W, which can include a waste container 172 and a vacuum or suction source (e.g., pump) 174, and/or any other components, devices, features, etc., is configured to receive and collect spent fluids or other treatment materials, exfoliated skin, sebum, blackheads and/or other debris removed by the handpiece 10 during use.

In other embodiments, as schematically depicted in FIG. 3B, the waste system W is positioned entirely or at least partially on and/or within the tower or console system 200. In some arrangements, one or more components of the waste system W can be configured to be secured to and removed from the tower or console system 200, as desired or required. For example, the waste bin or other container 172 can be removably securable to the tower or console (e.g., for emptying, cleaning, replacement, etc.). For example, fluid and/or other treatment material can flow from a fluid source 110 by a passage 120 through a flow control device 122, into a collection header 160, out the console 200 by a console exit 210 by a conduit 140 connected to the handpiece 10. Fluid and/or other treatment material can then continue from the handpiece 10 back through the console exit 210 back to the waste system W, into the waste bin 172, the fluid being drawn by a vacuum source 174.

FIG. 4 schematically illustrates a cross-sectional view of a tip 30 configured to secure along a distal end of a handpiece 10 of a skin treatment system. As shown, the tip 30 can include a peripheral or outer lip or member 32 along its distal end that is configured to contact skin tissue S during use. Once the tip 30 has been positioned relative to the subject's skin tissue S, at least a partial seal (or substantially a partial seal) can be established between the peripheral lip 32 and the adjacent skin tissue S. In some embodiments, the peripheral lip or similar distal member 32 can define an interior area 34 along the distal end or portion of the tip. Such an interior area 34 can be in fluid communication with one or more passageways 36, 38 of the tip 30. The passageways 36, 38 can include one or more fluid delivery passageways 36 that is/are configured to deliver treatment fluids, water or other dilutants and/or other fluids or materials being delivered via one or more fluid channels or pathways 22 of a handpiece 10. Such pathways 22 can be located at least partially within an interior of the handpiece 10, as illustrated in FIG. 4. However, in some arrangements, one or more fluid pathways 22 can be positioned along an exterior of the handpiece (e.g., along an outside of the housing 20 of the handpiece 10), either in lieu of or in addition to any internal pathways 22, as desired or required.

With continued reference to the schematic of FIG. 4, the tip can include one or more waste passageways 38 that is/are configured to transfer spent fluids and/or other treatment materials, exfoliated skin and other materials removed from the subject being treated (e.g., sebum, blackheads, etc.) and/or other debris and waste away from the tip 30. As discussed herein, such waste materials can be transferred to a waste container or canister of the system's waste collection system W. Similar to the fluid delivery channels or pathways 22 discussed above, the handpiece can include one or more waste channels or pathways 24. Such waste channels or pathways 24 can be located, at least partially, within an interior of the handpiece 10 (e.g., within an interior of a housing 20 of the handpiece, as illustrated schematically in FIG. 4) and/or along an exterior of the handpiece 10, as desired or required.

As discussed above, when a tip 30, such as the one schematically depicted in FIG. 4, is secured to a distal end of a handpiece 10, the In some embodiments, and/or other fluids or materials being delivered via one or more fluid pathways 22 of a handpiece 10. Such pathways 22 can be located at least partially within an interior of the handpiece 10, as illustrated in FIG. 4. However, in some arrangements, one or more fluid pathways 22 can be positioned along an exterior of the handpiece (e.g., along an outside of the housing 20 of the handpiece 10), either in lieu of or in addition to any internal pathways 22, as desired or required.

Once the tip 30 has been secured to a distal end of the handpiece 10, the fluid delivery channel(s) or pathway(s) 22 of the handpiece 10 can be placed in fluid communication with the delivery passageway(s) 36 of the tip 30. Likewise, the waste channel(s) or pathway(s) 24 of the handpiece 10 can be placed in fluid communication with the waste passageway(s) 38 of the tip 30. Thus, in some embodiments, when the peripheral lip or similar distal feature or member 32 of the tip 30 contacts skin tissue S and the waste collection system W (see, e.g., FIGS. 1, 3A and 3B) is activated, a vacuum force can be applied to the waste channel(s) or pathway(s) 24 of the handpiece 10. In turn, with the tip 30 secured to the handpiece 10, such a vacuum force will be applied to the waste passageway(s) 38 of the tip 30 and the interior area 34 located along the distal end or portion of the tip 30. If sufficient contact is made between the peripheral lip 32 and adjacent skin tissue S, a seal or a substantial seal can be established between the peripheral lip 32 and the adjacent skin tissue S. Accordingly, in such circumstances, the interior area 34 of the tip 30 and other passageway(s), channel(s) and/or other portions that are in fluid communication with the interior area 34 will also be subjected to a vacuum force created by a vacuum source (e.g., a pump) of the waste collection system. Such passageway(s) and channel(s) include, without limitation, the fluid delivery passageway(s) 36 of the tip and the fluid delivery channel(s) or pathway(s) 22 of the handpiece 10.

As a result, with the waste collection system activated, while spent treatment fluids and/or other treatment materials, exfoliated skin, sebum, blackheads and/or other debris or waste are transferred from the interior area 34 and other portions of the distal end of the tip 30, treatment fluids and/or other treatment materials can be advantageously simultaneously transferred or “pulled” from one or more treatment fluid or material sources (e.g., one or more bottles or other containers secured to a manifold assembly, a vial or container secured to the handpiece, etc.) to the tip 30 (e.g., via the fluid delivery channel(s) or pathway(s) 22 of the handpiece 10 and the fluid delivery passageway(s) 36 of the tip). In some arrangements, this is accomplished without the need to provide positive pressure or force to the delivery channels, pathways or passageways of the handpiece and tip. Thus, in some embodiments, treatment fluids and/or other treatment materials can be delivered to the tip at the same time that waste is being removed away from the tip 30 during use (e.g., using only the vacuum created by the vacuum or suction source of the waste collection system). FIG. 4 depicts one possible arrangement of a flow rate through the tip during operation. As shown, treatment material can be drawn (e.g., via suction) from the pathway 22 of the handpiece in flow path 23. Such treatment material can then enter the fluid delivery passageway 36 of the tip in flow path 37, and be applied to the targeted skin surface S. Excess treatment fluid along with various impurities extracted from the skin and/or other waste created during a skin treatment procedure can be removed from the tip by waste passageway 38 with flow path 39, and from the tip by waste channel 24 with flow path 25.

FIGS. 5A and 5B schematically illustrate a sealing cap 40 that can be secured to the distal end of a handpiece 10. In some embodiments, the sealing cap 40 is sized, shaped and otherwise configured to attach to the distal end of the handpiece 10 in an identical or similar manner than a tip 30 secured to the handpiece.

According to some embodiments, a sealing cap 40 is secured to the distal end of the handpiece 10 (as illustrated in FIGS. 5B and 5C) in order to place the fluid delivery and waste channels or pathways 22, 24 in fluid communication with one another. This can be done for a variety of reasons. For instance, as discussed in greater detail below, the sealing cap 40 can be secured to the distal end of the handpiece 10 and the waste system's vacuum source (e.g., vacuum pump) can be activated when a need exists to flush out the fluid delivery channel(s) or passageways(s) 22 of the handpiece and any upstream fluid components of the system. In some embodiments, such upstream fluid components of the system include, for example and without limitation, any treatment fluid delivery conduits 142 (see, e.g., FIGS. 1, 3A and 3B) placing the handpiece 10 in fluid communication with a manifold assembly 100, a manifold assembly 100, a purge system 300 and/or any other fluid conduits, passageways or components.

As schematically depicted in FIG. 5B, with the sealing cap 40 secured to the distal end of the handpiece 10 and the vacuum source (e.g., vacuum pump of the waste collection system) activated, any treatment fluids and/or other materials contained within the fluid delivery channel(s) or passageway(s) 22 of the handpiece 10 and upstream portions of the skin treatment system's fluid delivery network (e.g., the treatment fluid delivery conduit 142 (see, e.g., FIGS. 1, 3A and 3B), the manifold assembly 100 and/or any other fluid conduits, passageways or components can be advantageously cleared or flushed. In some embodiments, such fluids and/or other treatment materials are transferred through the fluid delivery channel(s) or passageway(s) 22 of the handpiece 10 to a cavity 44 defined within an interior of the sealing cap 40 and/or the interior area defined between the sealing cap and the distal end of the handpiece (see, e.g., FIG. 5B). One arrangement of such a clearing or flushing fluid flow pattern is generally represented by arrows FP in the schematic of the distal end of the handpiece depicted in FIG. 5B. Additional disclosure regarding the devices, systems and/or methods for clearing or flushing fluids and/or other treatment materials from a skin treatment system is provided below.

FIG. 5C illustrates one embodiment of a handpiece 10 with a sealing cap 40 secured to its distal end. As shown, the handpiece 10 can include a fluid flow control device 50 to permit an aesthetician or other user regulate the volume or amount of fluids and/or other treatment materials that are delivered to the tip (when the tip is secured to the handpiece instead of the sealing cap in preparation for a treatment procedure). The fluid flow control device 50, which can be incorporated into any embodiments of a handpiece disclosed herein or equivalents thereof, can be operatively coupled (e.g., directly or indirectly) to one or more valves or other flow control devices (e.g., located within the fluid delivery conduit or pathway 22 of the handpiece, located within the fluid delivery conduit 142, a conduit or passageway of the manifold assembly 100, etc.). In other embodiments, the fluid flow control device 50 can be configured to regulate the amount of vacuum or suction that is generated in the handpiece 10 (e.g., the waste channels or pathways 24 of the handpiece), either in addition to or in lieu of controlling one or more valves or other flow control devices included within the fluid network, as desired or required.

FIG. 6 illustrates one embodiment of a manifold assembly 100 configured to be included as part of a tower or console 200 (see, e.g., FIG. 2) of a skin treatment system. The manifold assembly 100 can comprise portions that are internal to the tower or console 200. However, in some embodiments, certain portions of the assembly 100 are configured to extend (e.g., be exposed) to an exterior of the tower or console. For instance, as shown in FIG. 2, the console or tower 200 can be configured such that treatment fluid bottles or other containers 110 can be secured to loading area of the manifold assembly 100 that extends to an exterior portion or area of the console or tower 200.

With continued reference to FIG. 6, the manifold assembly 100 can include a plurality of siphon tubes 120 that are sized, shaped and otherwise configured to reach to or near the bottom of the interior of a bottle or other container 110 secured at that specific station. In some embodiments, the siphon tubes 120 are the individual fluid conduits or passages discussed in reference to FIG. 1 above. The bottles or other containers 110 can be configured to be coupled to the corresponding station of the manifold assembly 100 using a threaded connection. For example, a bottle or other container 110 can include a threaded neck portion that is configured to rotatably engage a portion of the manifold assembly with a corresponding threaded pattern. In other embodiments, however, the bottles or other containers 110 are secured to corresponding stations of the manifold assembly using any other releasable securement method or technology, such as, for example, a press-fit or friction-fit connection, any other type of mechanical coupling and/or the like, as desired or required. Regardless of the exact manner in which bottles or other containers 110 are secured to the manifold assembly 100, once secured to the assembly, such bottles or other containers can be advantageously placed in fluid communication with the manifold assembly 100 and other components of the fluid network of a skin treatment system (e.g., a fluid delivery conduit 142, a handpiece, a waste collection system, etc.).

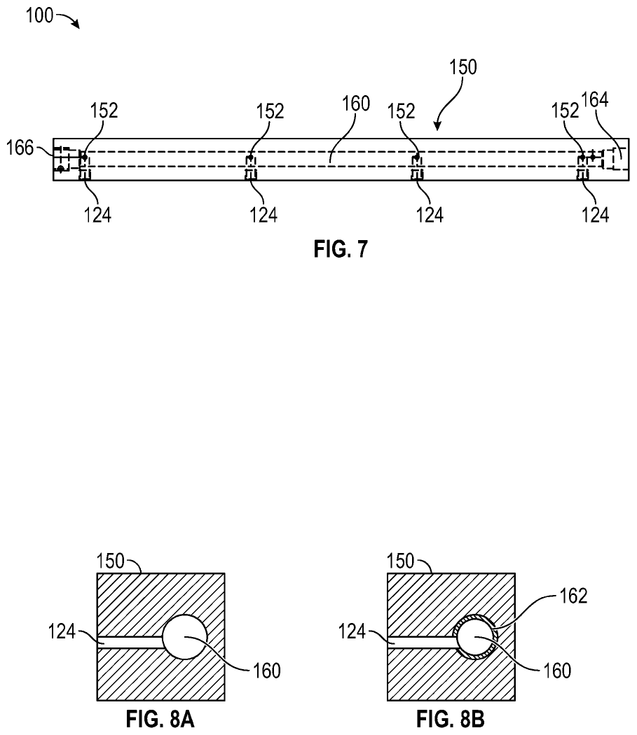

As discussed above and illustrated in the embodiment of FIGS. 6 and 7, the manifold assembly 100 can include a collection conduit or header 160 along a collection portion or zone 150. Such a collection conduit or header 160 can be configured to receive fluids and/or other treatment materials being transferred from one or more bottles or other containers 110 secured to the manifold assembly 100 to a handpiece that is placed in fluid communication with the manifold assembly 100 (e.g., via a fluid delivery conduit 142 and/or other intervening fluidic components). The transfer of fluids from the bottles 110 to the handpiece via the manifold assembly 100 can be accomplished by the application of vacuum along the distal end of the handpiece 10, as discussed in greater detail above. Valves or other fluid flow control devices or components 122 (see also, e.g., FIG. 1) positioned at each station of the manifold assembly 100 can selectively permit and prevent the transfer of fluids and/or treatment materials contained in the various bottles or other containers 110 secured to the different stations of the manifold assembly 100 to the collection conduit or header of the collection zone 150 of the assembly. In some embodiments, the system is configured to permit only sequential transfer of fluids or other materials from bottles or other containers secured to the manifold assembly. In other arrangements, however, the system is configured to permit parallel transfer of fluids (e.g., simultaneously transfer of fluids from two or more bottles 110 to the collection header).

With continued reference to FIG. 6, fluids and/or materials can be transferred from one or more bottles or other containers 110 secured to the manifold assembly 100 to one or more collection conduits or zones of the collection zone or portion 150 of the assembly 100. From there, fluids and/or other treatment materials are transferred to a distal or downstream end or portion 166 of the collection zone 150 before exiting an outlet portion 172 of the manifold assembly 100. This is generally represented by arrows 190 in FIG. 6. In some embodiments, the distal or downstream end of the outlet portion 172 can include a coupling (e.g., a luer, another type of standard or non-standard coupling, etc.) that is sized, shaped and otherwise configured to secure to a fluid delivery conduit 142 placing the manifold assembly 100 in fluid communication with a skin treatment handpiece 10. In other embodiments, an intermediate component (e.g., conduit, coupling, etc.) is included between the outlet portion 172 of the manifold assembly 100 and the fluid delivery conduit 142 (and/or other downstream components or portions of the skin treatment system's fluid network), as desired or required.

As illustrated in FIG. 6, the manifold assembly 100 can advantageously comprise a purge component or system 300. In the depicted embodiments, the purge system 300 is located along a proximal or upstream end or portion of the manifold assembly 100. In some arrangements, as discussed with reference to FIG. 1 and other depicted embodiments herein, the purge system 300 can include one or more valves or other flow control devices, components or features 310 that selectively open (e.g., expose) and close the purge assembly 300 to/from the ambient surroundings (e.g., the airspace in the room in which the skin treatment system is situated). The purge system 300, including the one illustrated in FIG. 6, can include a passive purge system or an active purge system, as desired or required by a particular application or use.

For any of the embodiments disclosed herein, the manifold assembly 100 can include a collection zone or portion 150 which comprises a single collection conduit or header 160. One embodiment of such a collection conduit or header 160 is illustrated in FIGS. 7, 8A and 8B. In some embodiments, the collection conduit or header 160 comprises an opening or passage that has been formed within a rigid member (e.g., a solid block or portion of metal, alloy and/or other material). In some embodiments, the collection conduit or header (e.g., an opening or passage 160) can include a single opening with a circular or substantially circular cross-sectional shape, as illustrated in FIGS. 8A and 8B. However, in other embodiments, the quantity of openings or passages, the cross-sectional shape of the opening and/or any other details of the collection conduit or header 160 can vary, as desired or required. For example, a manifold assembly 100 can include two or more (e.g., 2, 3, more than 3, etc.) individual collection conduits or headers 160 and/or the cross-sectional shape of the opening or passage 160 can be oval, square, other polygonal, irregular, otherwise non-circular, etc.).

In some embodiments, the collection zone 150 (e.g., header or opening 160) comprises one or more metals or alloys. By way of example and without limitation, the collection header or opening 160 can comprise aluminum. In other embodiments, however, the header or opening 160 can include one or more other metals or alloys, such as, for example various forms of aluminum (e.g., anodized aluminum), stainless steel, etc., either in addition to or in lieu of aluminum.

According to some arrangements, the collection zone of the manifold assembly 100 is manufactured using one or more manufacturing methods or techniques, such as, for example and without limitation, casting, molding, machining (or other removal of material from a larger bar, block or piece), extrusion, forging, deforming, stamping and/or any other fabrication method. In some embodiments, the header or opening 160 is machined from aluminum bar). The diameter or cross-sectional dimension of the opening 160 can be between 0.2 to 1.5 inches (e.g., 0.2 to 1.5, 0.2 to 0.5, 0.5 to 1, 1 to 1.5, 0.5 to 1.5, values between the foregoing ranges, etc.).

As illustrated in FIGS. 7, 8A and 8C, the collection zone or portion 150 of the manifold assembly 100 can include branches (e.g., smaller channels or passages) 124 that extend through a portion of the bar, block or other core portion 152 of the zone or portion to be placed in fluid communication with the header or opening 160. In some embodiments, such individual fluid branches 124 place the header or opening 160 in fluid communications with the internal contents of a corresponding bottle or other container 110 secured to a particular station of the manifold assembly 100, either directly or using one or more intermediate components, features, devices and/or the like (e.g., siphon tubes or other individual lines 120, valves or other fluid flow control devices 122, other conduits or passages, etc.).

In some embodiments, as depicted in FIG. 8B, at least a portion of the interior wall forming the collection conduit or header (e.g., opening or passage) 160 is provided with one or more layers, coatings or members 162. Such layers, coatings or members 162 can provide one or more benefits or advantages. For example, such components or features can reduce friction of fluid flow within the opening or passage 160 and facilitate the transfer of fluids and/or other materials therethrough. In addition, the use of such components or features can protect the metallic (e.g., aluminum) collection opening or passage 160. This can extend the life of the manifold assembly and reduce the likelihood of undesirable impacts on the assembly.

In some embodiments, the coating, layer or member 162 comprises a hydrophobic coating. The coating, layer or member 162 can comprise polytetrafluoroethylene (PTFE), perfluoroalkoxy alkane (PFA), fluorinated ethylene propylene (FEP), other fluoropolymers, acetal, another synthetic fluoropolymer of tetrafluoroethylene and/or any polymer or material. The coating, layer or member 162 can include an insert that is removably or fixedly attached to the interior of the collection conduit or passage 160. In some embodiments, the coating, layer or member 162 comprises a tube that is slidably delivered within the interior of the collection opening 160. In other embodiments, the coating, layer or member 162 is deposited along the interior of the collection conduit or passage 160 at the time of manufacture of the manifold assembly. For example, the coating can be applied via spray or other method before the manifold assembly is manufactured/assembled and used in the making of a skin treatment console or tower 200. One, two or more separate coatings, layers or other members can be used, as desired or required. The thickness of the coating, layer or member 162 is 0.1 to 3 mm (e.g., 0.1 to 3, 0.1 to 0.5, 0.5 to 1, 0.1 to 1, 0.1 to 1.5, 0.1 to 2, 0.1 to 2.5, 1 to 3, 1 to 2, 2 to 3 mm, values between the foregoing, etc.).

In some embodiments, a sleeve or other insert is positioned within the collection conduit or header (e.g., opening or passage) 160 and/or any other passages of a manifold or other fluid distribution system or sub-system. The manifold (and/or the console and/or other portions or components of the system) can be configured to remove and replace such a sleeve or other insert. Thus, the sleeve or insert can be removably (e.g., slidably removably) positioned within the header and/or other passage(s) of the manifold assembly. In other embodiments, the insert is fixedly or non-removably positioned with the heard and/or other passages. The insert can include one or more materials listed herein (e.g., see above in connection with a coating or layer) and can include any thickness and/or other dimension, including, without limitation, a thickness noted above. For instance, the thickness (e.g., wall thickness) of the insert or sleeve is 0.1 to 3 mm (e.g., 0.1 to 3, 0.1 to 0.5, 0.5 to 1, 0.1 to 1, 0.1 to 1.5, 0.1 to 2, 0.1 to 2.5, 1 to 3, 1 to 2, 2 to 3 mm, values between the foregoing, etc.).

Any other portion of the manifold assembly 100, including, for example and without limitation, the individual branches 124, can also comprise a similar coating, layer or member as the one used for the main collection header or passage 160, as desired.

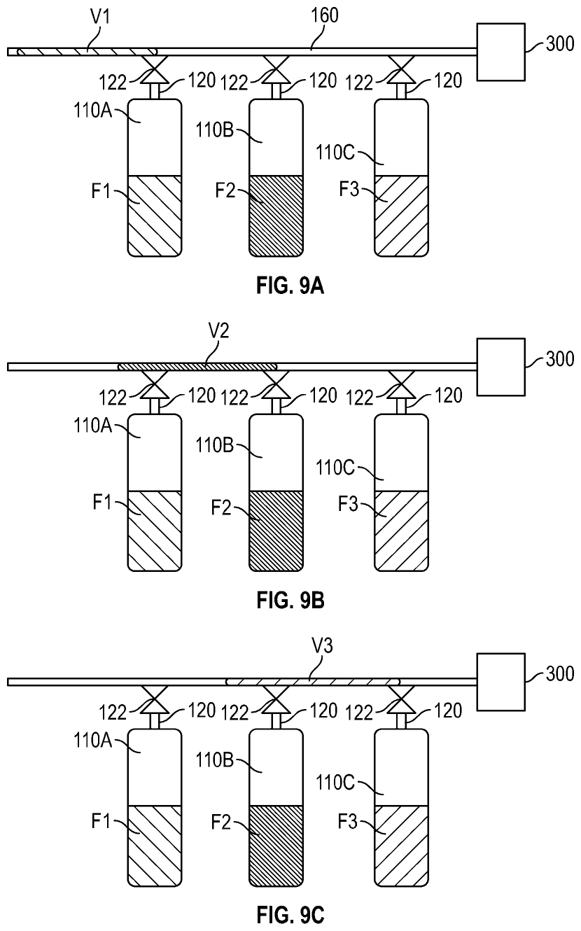

FIGS. 9A to 9B schematically illustrate various embodiments of fluid flow within a manifold assembly or other fluid distribution member or system 100 that includes a purge device or assembly 300. Certain fluid flow patterns are discussed below to provide non-limiting examples of the manifold assembly 100 in use. However, these embodiments are only examples, and as such, the various inventive concepts related to an enhanced manifold assembly and/or other aspects of a skin treatment system should not be limited in any way because of these examples.

With reference to FIG. 9A, a manifold assembly or other fluid distribution member or system 100 can include three stations or securement locations, each of which is configured to receive (e.g., removably) a bottle, container or other treatment fluid source 110A, 110B, 110C. In the depicted embodiments, the first bottle (“Bottle A”) 110A contains a first treatment fluid (“Fluid A”) F1, the second bottle (“Bottle B”) 110B contains a second treatment fluid (“Fluid B”) F2 and the third bottle (“Bottle C”) 110C contains a third treatment fluid (“Fluid C”) F3.

In FIG. 9A, a volume of Fluid A V1 remains in the collection header 160. This can be after the completion of a step (e.g., a non-final step) during the execution of a particular treatment procedure or it can be after the completion of the entire procedure. Regardless of when such a volume V1 remains in the collection header 160 and/or where the volume V1 should be delivered, in some arrangements, this volume of Fluid A V1 usually needs to be evacuated or otherwise eliminated from the collection header 160 prior to a subsequent step or procedure. If the manifold system 100 did not include a purge device or system 300, this treatment fluid volume V1 would typically be eliminated by attaching a sealing cap 40 to the distal end of the handpiece 10 and activating the vacuum source of the waste collection system to transfer the volume V1 from the collection header or conduit 160, in sequence, to the distal end of the collection header 166 (FIG. 7), out of the distal end or portion of the manifold assembly 172 (FIG. 6), through one or more fluid delivery conduits 142, through any fluid delivery conduits or passages 22 of the handpiece 10, to and through cavity 44 formed by the sealing cap 40, through the waste conduits or passages 24 of the handpiece, through one or more waste conduits 144 secured to the handpiece and to the waste collection system (e.g., the waste bin or canister 172) W.

Accordingly, in such embodiments, the volume V1 remaining within the collection header and likely or possibly in various other portions of the system (e.g., the distal or downstream portion or section 190 of the manifold assembly, other parts of the manifold assembly 100, the fluid delivery conduit 142, the fluid delivery conduit(s) or passage(s) 22 of the handpiece 10, etc.) is wasted and not used. This can increase, sometimes significantly, the volume of treatment materials that is wasted or otherwise not delivered to the skin surfaces of subjects undergoing treatment. Not only can this make skin treatment procedures more costly, but it can unnecessarily increase the amount of waste that is produced during microdermabrasion and/or other skin treatment procedures.

In some embodiments, the incorporation of a purge system 300 can meaningfully reduce the amount of treatment fluid that is wasted. Further, this can provide one or more related or other benefits and advantages. For instance, currently, the amount of treatment fluid that is wasted because a skin treatment system's manifold assembly does not include a purge system 300 can be 20% to 200% (e.g., 20 to 50, 50 to 100, 80 to 120, 50 to 150, 100 to 120, 100 to 150, 100 to 200, 20 to 200%, percentages between the foregoing ranges, etc.) of the volume of the treatment fluid that is used in a procedure. In one embodiment, using a manifold system 100 with a purge system 300, e.g., as disclosed herein, can reduce the amount of treatment fluid that is wasted by approximately 100% (e.g., 50 to 150%, 75 to 125%, 20 to 200%, more than 200%, etc.). By way of example, the volume of treatment liquid that is used for priming a system without a purge system is approximately 12 ml. In one such embodiment, the volume of treatment liquid that is delivered to the subject's skin during a procedure is approximately 12 ml. Thus, in some arrangements, the total volume of treatment liquid used during a procedure can be reduced by 100% or substantially 100% (or more) relative to devices/systems that do not include a purge system.

As discussed, the purge system 300 can be located along a proximal or upstream end or portion 164 of the collection header or conduit 160 of the manifold assembly 100. In some embodiments, as depicted in FIG. 6, the purge system 300 is connected to the upstream end or portion 164 of the collection header 160 using one or more intermediate conduits or portions 170. However, in alternative arrangements, the purge system 300 is directly or substantially directly coupled to the upstream end or portion 164 of the collection header 160. As discussed herein, the purge system 300 can include an active system in which the system 300 is configured to deliver air or gas through the downstream components of the manifold system 100 (e.g., the collection header 160, the fluid delivery conduit ‘162, fluid delivery passages 22 within the handpiece 10, etc.) to evacuate any fluid remaining in such components or portions. In other embodiments, the purge system 300 includes a passive system that relies on utilizing the vacuum or suction that is generated by a vacuum pump of the waste collection system to generate the necessary pressure differential in the manifold assembly and other hydraulic components. In some arrangements, the purge system 300 can be configured to be used both as a passive system or an active system, at the direction of the user, the overall system and/or any other factor or consideration.

A passive purge system 300 can include one or more valves or other fluid flow control devices that selectively place the manifold assembly 100 and its various hydraulic portions and components (e.g., the collection header 160) in fluid communication with the ambient surroundings. Such valves 310 can be automatically and remotely controlled in accordance with a control scheme of the skin treatment system. In some embodiments, the valves can be manually and/or locally controlled, cither in addition to (e.g., as a manual override) or in lieu of automatic and/or remote control.

In some embodiments, the purge assembly 300 comprises one or more compressors or other devices configured to generate air or gas at a pressure above atmospheric.

Regardless of its exact design and configuration, the purge system 300 can permit any fluid remaining in the collection header 160, other portions of the manifold 100 and/or any other hydraulic components that help transfer treatment fluids and/or other materials from the bottles or other containers 110A, 110B, 110C secured to the manifold assembly to the handpiece 10. For example, when the purge system 300 is activated (e.g., by opening a valve 310 of the system 300 and exposing the system and downstream portions of the manifold assembly 100 to the surrounding atmosphere), activation of the waste system's vacuum pump can permit any volume of treatment fluids V1, V2, V2 (in FIGS. 9A to 9C) trapped within the collection header 160 to be delivered to the handpiece, and thus to the skin surface being treated. This is not possible without a purge system, as the manifold system does not have a way of accepting air or other gas to allow the vacuum created within the passages of the manifold to move the trapped treatment fluid volume to the handpiece. Instead, in such systems, a rinsing solution is used (which can be contained in a bottle or other container 110 secured to one of the manifold system's stations) to expunge this volume of trapped treatment fluid from the manifold assembly 100 and other components of the system's fluid delivery hydraulic network. This can further add to the amount of waste volume that is generated during the execution of a treatment procedure.

According to some embodiments, the treatment system is configured to sequentially deliver treatment fluids from two or more (e.g., 2, 3, 4, etc.) different bottles or containers 110A, 110B, 110C secured to the manifold assembly 100. In the embodiments schematically illustrated in FIGS. 9A to 9C, the volume of fluid F1, F2, F3 that is transferred out of each bottle or container 110A, 110B, 110C is automatically and carefully determined by the skin treatment system (e.g., the processor of the system) based on what will be delivered to the subject's skin surface. Unlike skin treatment systems that do not utilize a purge system in their manifold assemblies, the volume of any additional treatment fluid will be reduced or eliminated. As discussed, skin treatment systems without purge systems are required to transfer a volume of treatment fluid that is greater than what will actually be delivered to the subject. For example, the volume of fluid V1 in FIG. 9A remains in the collection header 160 and other portions of the manifold assembly and other hydraulic components of the system after the desired volume of treatment volume has already been delivered to the subject, as there is no alternative method of transferring the desired amount of treatment fluid to the subject without having a trapped fluid volume that remains.

Therefore, in some embodiments, the system's processor is able to determine exactly how much volume should be transferred to the skin. Only that volume of treatment fluid from each bottle or other container is moved out of from the bottle or other container. The use of the purge system 300 can help move that entire volume or slug to the handpiece and to the subject being treated, thereby reducing or eliminating waste. This can be repeated for subsequently delivered fluids, e.g., fluids F2, F2 contained in bottles 110B, 110C. Treatment fluids can be delivered to the handpiece and the subject's skin surface sequentially, with or without an intervening rising or cleaning step.

For any of the manifold assembly or other fluid distribution system embodiments disclosed herein, the purge system can include one or more components or features to enhance its operation. For example, the purge system 300 can include a muffler or noise dissipating devices, portions, components and/or features. Further, the purge system can include one or more filters to ensure that any ambient air entering the purge system 300 (and thus, the manifold assembly and other downstream components of the skin treatment's hydraulic network) are clean and will not damage the system.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods summarized above and set forth in further detail below describe certain actions taken by a practitioner; however, it should be understood that they can also include the instruction of those actions by another party. The methods summarized above and set forth in further detail below describe certain actions taken by a user (e.g., a professional in some instances); however, it should be understood that they can also include the instruction of those actions by another party. Thus, actions such as “moving a handpiece” or “delivering a fluid” include “instructing moving a handpiece” and “instructing delivering a fluid.” The ranges disclosed herein also encompass any and all overlap, sub-ranges, and combinations thereof. Language such as “up to,” “at least,” “greater than,” “less than,” “between,” and the like includes the number recited. Numbers proceeded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

What is claimed is:

1. A skin treatment system comprising:
at least two stations, each of the at least two stations being configured to receive a fluid container, wherein the at least two stations are included in a manifold assembly;
a single fully-enclosed collection header; and
at least one fluid connector configured to place treatment material included in the fluid containers secured to the stations in fluid communication with the collection header;
wherein a downstream end of the collection header is configured to be in fluid communication with a fluid conduit that places the collection header in fluid communication with a handpiece.

2. The system of claim 1, wherein the collection header is machined from a larger member comprising at least one metal and/or alloy.

3. The system of claim 2, wherein the at least one metal and/or alloy comprises aluminum.

4. The system of claim 1, wherein the collection header is configured to be placed in fluid communication with a purge system.

5. The system of claim 1, wherein an upstream end of the collection header is configured to be placed in fluid communication with a purge system.

6. The system of claim 4, wherein the purge system is configured to permit the collection header to be selectively purged or substantially purged of any treatment material.

7. The system of claim 6, wherein the purge system comprises a valve or other flow control device that is configured to place the collection header in fluid communication with an ambient space.

8. The system of claim 4, wherein the purge system comprises a pump, compressor or other device configured to create a positive pressure relative to an ambient pressure within the collection header.

9. The system of claim 4, wherein the purge system does not comprise a pump, compressor or other device configured to create a positive pressure relative to an ambient pressure within the collection header.

10. The system of claim 1, further comprising a vacuum source, wherein the vacuum source is configured to create a vacuum or negative pressure relative to an ambient pressure within the collection header.

11. The system of claim 1, wherein the manifold assembly is included in a tower or console of the system.

12. The system of claim 1, further comprising a user interface.

13. The system of claim 12, wherein the user interface comprises a touchscreen device or component.

14. The system of claim 1, further comprising a handpiece configured to be placed in fluid communication with the collection header and other portions of the manifold assembly.

15. The system of claim 1, wherein the manifold assembly comprises at least three stations.

16. The system of claim 1, wherein the manifold assembly comprises at least four stations.

17. The system of claim 1, wherein the collection header comprises a circular or oval cross-sectional shape.

18. The system of claim 1, wherein the collection header comprises a single conduit or passage.

19. The system of claim 1, wherein an interior of the collection header comprises at least one layer, coating, lining, sleeve, insert or member.

20. The system of claim 19, wherein the at least one layer, coating, lining or member reduces friction of fluid flow through the collection header.

21. The system of claim 19, wherein the at least one layer, coating, lining or member offers protection to the collection header.

22. The system of claim 19, wherein the at least one layer, coating, lining or member is hydrophobic.

23. The system of claim 22, wherein the at least one layer, coating, lining or member comprises at least one of the following: polytetrafluoroethylene (PTFE), perfluoroalkoxy alkanes (PFA), fluorinated ethylene propylene (FEP), another fluoropolymers, acetal, another synthetic fluoropolymer of tetrafluoroethylene and/or any polymer or material.

24. The system of claim 19, the at least one layer, coating, lining or member comprises an insert that is removably or fixedly attached to an interior of the collection header.

25. The system of claim 19, the at least one layer, coating, lining or member is deposited or otherwise provided along an interior of the collection header at the time of manufacture of the system.

26. A skin treatment system comprising:
at least two stations, each of the at least two stations being configured to receive a fluid container, wherein the at least two stations are included in a manifold assembly;
a collection header;
wherein treatment material included in the fluid containers secured to the stations is in fluid communication with the collection header; and
wherein a downstream end of the collection header is configured to be in fluid communication with a fluid conduit that places the collection header in fluid communication with a handpiece; and
a purge system configured to permit the collection header to be selectively purged or substantially purged of any treatment material.

27. The system of claim 26, wherein an upstream end of the collection header is configured to be placed in fluid communication with a purge system.

28. The system of claim 26, wherein the purge system comprises a valve or other flow control device that is configured to place the collection header in fluid communication with an ambient space.

29. The system of claim 26, wherein the purge system comprises a pump, compressor or other device configured to create a positive pressure relative to an ambient pressure within the collection header.

30. The system of claim 26, wherein the purge system does not comprise a pump, compressor or other device configured to create a positive pressure relative to an ambient pressure within the collection header.

31. The system of claim 26, further comprising a vacuum source, wherein the vacuum source is configured to create a vacuum or negative pressure relative to an ambient pressure within the collection header.

32. The system of claim 26, wherein the manifold assembly is included in a tower or console of the system.

33. The system of claim 26, further comprising a user interface.

34. The system of claim 33, wherein the user interface comprises a touchscreen device or component.

35. The system of claim 26, further comprising a handpiece configured to be placed in fluid communication with the collection header and other portions of the manifold assembly.

36. The system of claim 26, wherein the collection header comprises a single fully-enclosed collection header.

37. The system of claim 26, wherein the collection header is machined from a larger member comprising at least one metal and/or alloy.

38. The system of claim 37, wherein the at least one metal and/or alloy comprises aluminum.

39. The system of claim 26, wherein the collection header comprises a single conduit or passage.

40. The system of claim 26, wherein an interior of the collection header comprises at least one layer, coating, lining or member.

41. The system of claim 40, wherein the at least one layer, coating, lining or member reduces friction of fluid flow through the collection header.

42. The system of claim 40, wherein the at least one layer, coating, lining or member offers protection to the collection header.

43. The system of claim 40, wherein the at least one layer, coating, lining or member is hydrophobic.

44. The system of claim 43, wherein the at least one layer, coating, lining or member comprises at least one of the following: polytetrafluoroethylene (PTFE), perfluoroalkoxy alkanes (PFA), fluorinated ethylene propylene (FEP), another fluoropolymers, acetal, another synthetic fluoropolymer of tetrafluoroethylene and/or any polymer or material.

45. The system of claim 40, the at least one layer, coating, lining or member comprises an insert that is removably or fixedly attached to an interior of the collection header.

46. The system of claim 40, the at least one layer, coating, lining or member is deposited or otherwise provided along an interior of the collection header at the time of manufacture of the system.
